(19) United States
(12) Patent Application Publication
(10) Pub. No.: US 2015/0376525 A1
(43) Pub. Date: Dec. 31, 2015

(54) SELECTIVE RECOVERY OF C2+ HYDROCARBONS FROM NATURAL GAS FOR STEAM CRACKER FEED IN AN INTEGRATED REFINERY AND STEAM CRACKER COMPLEX USING PRESSURE SWING ADSORPTION

(71) Applicant: UOP LLC, Des Plaines, IL (US)

(72) Inventors: Robert E. Tsai, Arlington Heights, IL (US); Ronald J. Long, Arlington Heights, IL (US); Xin X. Zhu, Long Grove, IL (US); Kirit M. Patel, Winfield, IL (US)

(21) Appl. No.: 14/747,603

(22) Filed: Jun. 23, 2015

Related U.S. Application Data
(60) Provisional application No. 62/017,823, filed on Jun. 26, 2014.

(51) Int. Cl. C10L 3/10; C07C 7/13; B01D 53/047; C07C 4/04

(52) U.S. Cl. CPC C10L 3/10; C07C 4/04; C07C 7/13; B01D 53/047; C10L 2290/541; C10L 2200/0277; B01D 2253/102; B01D 2253/104; B01D 2253/106; B01D 2253/116; B01D 2256/245; B01D 2256/10; B01D 2257/702; B01D 2256/16

(57) ABSTRACT

The invention provides for sending a natural gas stream through a pressure swing adsorption unit to send a gas stream comprising mainly nitrogen, methane and hydrogen to a fuel gas stream and a gas stream comprising a significant majority of C₂+ hydrocarbons from the natural gas stream to a tail gas stream to then go to a stream cracker.

### CROSS-REFERENCE TO RELATED APPLICATION

This application claims priority from Provisional Application No. 62/017,823 filed Jun. 26, 2014, the contents of which are hereby incorporated by reference.

### BACKGROUND OF THE INVENTION

Steam crackers convert hydrocarbon feedstock to streams rich in light alkenes like ethylene and propylene and are used as a principal industrial means to generate these valuable petrochemical products. Fuel gases typically contain a small amount of C₂+ hydrocarbon content and therefore could have value as steam cracker feed; however, the remaining (majority) composition consists of lower-value components, such as nitrogen (N₂) and methane (C₁). Their presence can negatively impact the operation of the steam cracker, as they pass through unprocessed and thus waste capacity and energy. If the C₂+ hydrocarbons can be salvaged while minimizing the recovery of these other species, the corresponding capital and operating expense penalties can be limited—not just for the steam cracker but also for downstream low-temperature separation equipment.

A refinery gas having a composition similar to natural gas is provided in the present invention. The stream (“natural gas”) enters the complex at relatively high pressure (2377 kPa, 330 psig) and is used as part of the fuel gas pool in the prior art, although it notably has some C₂+ hydrocarbon material (around 10 mol %) that would be quite valuable as steam cracker feed.

### SUMMARY OF THE INVENTION

The invention provides a process for treating a natural gas stream comprising sending a natural gas stream to at least one pressure swing adsorption (PSA) unit comprising silica gel and/or a molecular sieve, separating the natural gas stream into a fuel gas stream and a tail gas stream, wherein the fuel gas stream comprises a higher concentration of methane and nitrogen than the natural gas stream and said tail gas stream comprises a higher concentration of C₂+ hydrocarbons than said natural gas stream. The tail gas stream may be sent to a steam cracker to be converted into light alkenes including ethylene and propylene or it may be first sent to a gas recovery unit (“gas plant”) for separation into two or more streams and then to the steam cracker. The tail gas stream may comprise at least 90% of the C₂+ hydrocarbons from the natural gas stream. The fuel gas stream may comprise at least 65% of the methane from the natural gas stream. Hydrogen may also be separated from the natural gas stream together with the methane and nitrogen.

### BRIEF DESCRIPTION OF THE DRAWING

FIG. 1 shows a flow scheme in which nitrogen and methane are concurrently separated by a PSA unit from a natural gas stream.

FIG. 2 shows an alternative flow scheme in which the tail gas stream from the PSA unit is sent to a gas plant and one or more streams from the plant are sent to a steam cracker.

### DETAILED DESCRIPTION OF THE INVENTION

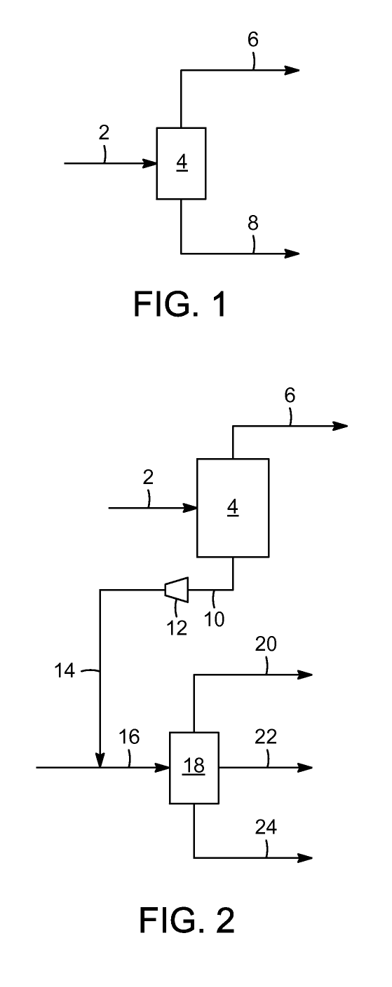

This invention involves an innovative PSA process to remove both nitrogen and methane from the natural gas and afterward send a stream enriched in C₂+ hydrocarbons to the steam cracker. In the new flow scheme of the invention (FIG. 1), the natural gas is sent directly to a PSA unit, which operates to separate out both nitrogen and methane while maximizing the recovery of C₂+ hydrocarbons to the tail gas. The low-pressure tail gas, which is consequently depleted in nitrogen and methane, is then fed to the steam cracker. The high-pressure PSA product stream consists of nitrogen and methane, along with small quantities of C₂+ hydrocarbons and can be used for fuel gas. Product value margin is greatly enhanced with the increased C₂+ hydrocarbons in the feed to the steam cracker and at the same time, capital and operating expense penalties are minimized by minimizing the nitrogen and methane in the feed. A significant net product value gain can be achieved with the FIG. 1 configuration as compared to the prior art process of allowing the C₂+ hydrocarbons to be sent to fuel gas.

A change to the base scheme is shown in FIG. 1. The new idea basically shifts a significant amount of the natural gas over to steam cracker feed. A novel PSA process is utilized to “pre-treat” this feed, wherein a large portion of nitrogen and methane are stripped out. Hydrogen may also be stripped out as well. As much as all of the C₂+ hydrocarbons in the natural gas can therefore be sent to the steam cracker. The following assessment is based on the diagram in FIG. 1, but other conditions and configurations are possible. For instance, an alternative flow scheme is shown in FIG. 2. The PSA tail gas is compressed and combined with refinery off gases (e.g., stripper column off gases from various hydroprocessing units, or “stripper gases”) and processed in a gas plant that includes a number of columns and other separation apparatus to separate the gases by their number of carbon atoms. In a typical configuration, separate products are generated from the gas plant: naphtha, LPG (C₃/C₄ material), and lean gas containing mostly hydrogen and C₂− hydrocarbons. These streams could all eventually feed into the steam cracker, so this flow scheme could be considered if it is infeasible or undesirable to send the PSA tail gas directly there, as in FIG. 1. Furthermore, only a single gas feed (“natural gas”) is referenced, but this process could accept streams of comparable composition from throughout the refinery complex, as a centralized separation section.

This invention provides a means to recover C₂+ material as steam cracker feed from a natural gas-type stream (rich in nitrogen/methane) without concurrently overloading the steam cracker with these components (nitrogen/methane). The integration of at least one PSA unit into the flow scheme creates significant business value.

PSA provides an efficient and economical means for separating a multi-component gas stream containing at least two gases having different adsorption characteristics. The more strongly adsorbable gas can be an impurity which is removed from the less strongly adsorbable gas which is taken off as product; or, the more strongly adsorbable gas can be the desired product, which is separated from the less strongly adsorbable gas. In PSA, a multi-component gas is typically fed to at least one of a plurality of adsorption zones at an elevated pressure effective to adsorb at least one component, while at least one other component passes through. At a defined time, the feed stream to the adsorber is terminated and the adsorption zone is depressurized by one or more co-current depressurization steps wherein pressure is reduced to a defined level which permits the separated, less strongly adsorbed component or components remaining in the adsorption zone to be drawn off without significant concentration of the more strongly adsorbed components. Then, the adsorption zone is depressurized by a counter-current depressurization step wherein the pressure on the adsorption zone is further reduced by withdrawing desorbed gas counter-currently to the direction of the feed stream. Finally, the adsorption zone is purged and repressurized. The combined gas stream produced during the counter-current depressurization step and the purge step is typically referred to as the tail gas stream. The final stage of repressurization is typically performed by introducing a slipstream of product gas comprising the lightest gas component produced during the adsorption step. This final stage of repressurization is often referred to as product repressurization. In multi-zone systems, there are typically additional steps, and those noted above may be done in stages. Various classes of adsorbents are known to be suitable for use in PSA systems, the selection of which is dependent upon the feedstream components and other factors. Molecular sieves such as the microporous crystalline zeolite and non-zeolitic catalysts, particularly aluminophosphates (AlPO) and silicoaluminophosphates (SAPO), are known to promote reactions such as the conversion of oxygenates to hydrocarbon mixtures.

In FIG. 1, there is shown a natural gas stream 2 that is 8 mol % nitrogen, 81 mol % methane and 10 mol % C₂+ hydrocarbons. The estimated flow rate of natural gas stream 2 is 100 MT/day, 5.2 MT-mole/day and 0.125 MMSCMD (4.4 MMSCFD). Natural gas stream 2 is sent to a PSA unit 4 that separates nitrogen and methane, as well as hydrogen, preferentially from C₂+ hydrocarbons. In fuel gas stream 6 there is 11 mol % nitrogen, 89 mol % methane and 0.1 mol % C₂+ hydrocarbons. The estimated flow rate of fuel gas stream 6 is 54 MT/day, 3.1 MT-mole/day and 0.074 MMSCMD (2.6 MMSCFD). Tail gas stream 8 contains the vast majority of the C₂+ hydrocarbons with an approximate composition in this example of 5 mol % nitrogen, 70 mol % methane and 25 mol % C₂+ hydrocarbons. The estimated flow rate of tail gas stream 8 that is sent to a stream cracker is 46 MT/day and 2.1 MT-mole/day. An additional PSA unit may be provided to further concentrate the C₂+ hydrocarbons stream to the steam cracker and to further concentrate the methane being sent to fuel gas.

The tail gas at low pressure may be sent to the suction of a cracked gas compressor which compresses the gases from the steam cracker furnaces prior to being sent to the product recovery section (pre-treating, cold box and fractionation). The product recovery section will recover the C₂+ paraffin material that is recycled to the steam cracker furnaces. The methane and nitrogen will be separated out by the cold box in the product recovery section. An alternative arrangement is to feed the tail gas product directly to the steam cracker furnaces. This could be done by compressing the tail gas to the pressure required to get it into the furnaces or by designing the PSA tail gas with a pressure sufficient to get it into the steam cracker furnaces. The steam cracker furnace products will then go to the cracked gas compressor and be processed as discussed above.

In FIG. 2, there is shown an alternative flow scheme for processing the natural gas in which instead of the tail gas going to a steam cracker, it is sent to a gas plant to undergo further separation such as separation into C₁, C₂, C₃, etc. streams. A natural gas stream 2 is sent to a PSA unit 4 that provides a fuel gas stream that is mainly methane, nitrogen and hydrogen and a gas stream 10 that has the vast majority of C₂+ hydrocarbons. Gas stream 10 is shown passing through a compressor 12 to a compressed stream 14 that is combined with a stripper gas blend 16 and then to a gas plant 18 that consists of a series of absorbers, fractionators, or other separation apparatus. Other refinery streams (not shown in FIG. 2) such as unstabilized naphtha or lean oil are also generally sent to the plant to participate in the separation. In typical configurations, the plant produces at least three streams, a lean gas stream 20, an LPG stream 22 and a naphtha stream 24.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention. In other words, the present invention may be embodied in other specific forms without departing from its spirit or essential characteristics. The described implementations are to be considered in all respects only as illustrative and not restrictive. The scope of the invention should, therefore, be determined not with reference to the above description, but instead should be determined with reference to the pending claims along with their full scope or equivalents, and all changes which come within the meaning and range of equivalency of the claims are to be embraced within their full scope.

### SPECIFIC EMBODIMENTS

While the following is described in conjunction with specific embodiments, it will be understood that this description is intended to illustrate and not limit the scope of the preceding description and the appended claims.

A first embodiment of the invention is a process for treating a natural gas stream comprising (a) sending a natural gas stream to at least one pressure swing adsorption unit comprising silica gel and/or a molecular sieve; and (b) separating the natural gas stream into a fuel gas stream and a tail gas stream, wherein the fuel gas stream comprises a higher concentration of methane, nitrogen and hydrogen than the natural gas stream and the tail gas stream comprises a higher concentration of C₂+ hydrocarbons than the natural gas stream. An embodiment of the invention is one, any or all of prior embodiments in this paragraph up through the first embodiment in this paragraph wherein the tail gas stream is sent to a steam cracker. An embodiment of the invention is one, any or all of prior embodiments in this paragraph up through the first embodiment in this paragraph wherein the tail gas stream is first sent to a gas plant and one or more of the streams from the plant are sent to the steam cracker. An embodiment of the invention is one, any or all of prior embodiments in this paragraph up through the first embodiment in this paragraph wherein the tail gas stream comprises at least 90% of the C₂+ hydrocarbons from the natural gas stream. An embodiment of the invention is one, any or all of prior embodiments in this paragraph up through the first embodiment in this paragraph wherein the fuel gas stream comprises at least 65% of the methane from the natural gas stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for treating a natural gas stream comprising:
(a) sending a natural gas stream to at least one pressure swing adsorption unit; and
(b) separating said natural gas stream into a fuel gas stream and a tail gas stream, wherein the fuel gas stream comprises a higher concentration of methane and nitrogen than said natural gas stream and said tail gas stream comprises a higher concentration of C₂+ hydrocarbons than said natural gas stream.

2. The process of claim 1 wherein said tail gas stream is sent to a steam cracker.

3. The process of claim 2 wherein said tail gas stream is first sent to a gas plant and then to said steam cracker.

4. The process of claim 1 wherein said tail gas stream comprises at least 90% of the C₂+ hydrocarbons from said natural gas stream.

5. The process of claim 1 wherein said fuel gas stream comprises at least 65% of the methane from said natural gas stream.

6. The process of claim 1 wherein the pressure swing adsorption unit uses one or more of selective adsorbents selected from silica gel, activated carbon, activated alumina, or molecular sieves.

7. The process of claim 1 wherein said fuel gas stream further comprises hydrogen.
